(19) United States
(12) Patent Application Publication
(10) Pub. No.: US 2022/0130047 A1
(43) Pub. Date: Apr. 28, 2022

(54) DIAGNOSTIC IMAGING FOR DIABETIC RETINOPATHY

(71) Applicant: COMMONWEALTH SCIENTIFIC AND INDUSTRIAL RESEARCH ORGANISATION, Acton, Australian Capital Territory (AU)

(72) Inventors: Sajib SAHA, Acton (AU); Di XIAO, Acton (AU); Yogensan KANAGASINGAM, Acton (AU)

(21) Appl. No.: 17/429,076

(22) PCT Filed: Feb. 4, 2020
(86) PCT No.: PCT/AU2020/050080

§ 371 Date: Aug. 6, 2021

(30) Foreign Application Priority Data
Feb. 7, 2019 (AU) 2019900390

(51) Int. Cl. G06T 7/00; A61B 3/12; A61B 5/00; A61B 5/103; G06T 7/33; G06T 7/90; G06T 7/11; G06T 7/136

(52) U.S. Cl. CPC G06T 7/0016; G06T 2207/20081; A61B 5/4842; A61B 5/1032; A61B 5/7275; A61B 5/7267; G06T 7/33; G06T 7/90; G06T 7/11; G06T 7/136; A61B 2576/02; G06T 2207/10024; G06T 2207/30041; G06T 2207/20021; A61B 3/12

(57) ABSTRACT

This disclosure relates to diagnostic imaging of a retina of a patient with diabetic retinopathy. A processor retrieves a first image of the retina captured at a first point in time and a second image of the retina captured at a second point in time after the first point in time. The processor aligns the first image to the second image to reduce an offset between non-pathologic retina features in the first image and the second image and obtains image objects related to diabetic retinopathy in the first image and the second image. The processor then calculates a numerical pathology score indicative of a progression of the diabetic retinopathy by calculating a degree of change of the image objects related to diabetic retinopathy between the aligned first and second images and finally, creates an output representing the calculated numerical pathology score.

### CROSS-REFERENCE TO RELATED APPLICATIONS

The present application claims priority from Australian Provisional Patent Application No 2019900390 filed on 7 Feb. 2019, the contents of which are incorporated herein by reference in their entirety.

### TECHNICAL FIELD

This disclosure relates to diagnostic imaging for diabetic retinopathy.

### BACKGROUND

Diabetic retinopathy (DR) is a microvascular complication of diabetes, causing abnormalities in the retina and is one of the leading causes of blindness in the world. Early detection, and proper care and management is the key to overcome blindness from DR. Clinical signs observable in DR include microaneurysms, haemorrhages, exudates and intra-retinal micro-vascular abnormalities. While considerable research has been done on detecting DR pathologies from a single timestamp image, research to analyse the progression or regression of DR over time is still significantly limited. There is a need for automated, and quantitative approaches to detect the appearance of pathologic features, and to classify DR related changes in the retina over time.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

There is provided a method for diagnostic imaging where two images that have been captured over a period of time, such as one year, are used to quantitatively measure the progression of the retinopathy. In particular, the two images are aligned using non-pathologic retina features, such as blood vessels. This ensures that an offset error between the images does not corrupt the quantitative measurement of pathologic features, such as image objects related to diabetic retinopathy, when they are compared between the two images. For example, an increase in the area of a microaneurysm by one single pixel between doctor visits can be detected when the images are aligned accurately.

A method for diagnostic imaging of a retina of a patient with diabetic retinopathy comprises:

retrieving a first image of the retina captured at a first point in time, the first image being a photographic colour image;

retrieving a second image of the retina captured at a second point in time after the first point in time, the second image being a photographic colour image;

aligning the first image to the second image to reduce an offset between non-pathologic retina features in the first image and the second image;

obtaining image objects related to diabetic retinopathy in the first image and the second image;

calculating a numerical pathology score indicative of a progression of the diabetic retinopathy by calculating a degree of change of the image objects related to diabetic retinopathy between the aligned first and second images; and

creating an output representing the calculated numerical pathology score.

It is an advantage that the method calculates a numerical pathology score that is indicative of a progression of the disease. This way, a clinician or health care provider can obtain an meaningful number that clearly shows how the disease progresses in a quantitative way. The predictive future pathology map visualizes the areas with significant changes in colour over time. These allows the monitoring of the efficacy of medication and other therapeutic methods. The accuracy of the numerical pathology score, and predictive map computation is increased by using the non-pathologic retina features to align the images with sub-pixel accuracy. An accurate alignment supports the calculation of a numerical pathology score that is based on a degree of change of image objects. In other words, small inaccuracies in alignment lead to large inaccuracies in the numerical pathology score. Accurate alignment also plays an important role to generate the predictive future pathology map more accurately which is based on pixel-wise colour difference between images.

The degree of change may be indicative of a change in the number of image objects related to diabetic retinopathy. The degree of change may be indicative of a change in the area covered by image objects related to diabetic retinopathy present in the first image and the second image. The method may further comprise identifying areas on the image that are likely to develop pathology in future by determining a colour difference between the first image and the second image.

The output may comprise a predictive map with colour codes representing areas on the second image that showed changes in colour in comparison to the first image, the colour map being indicative of which areas are likely to develop pathology in future.

Aligning the first image to the second image may comprise detecting corresponding points in the non-pathologic retina features and reducing an offset between the corresponding points.

It is an advantage that corresponding points in the non-pathologic retina features do not change significantly over time. Therefore, the alignment can be performed accurately.

Detecting corresponding points may comprise calculating image features for the first and second images by grouping, for each image feature, pixels into a first group and a second group and calculating the image features based on a first aggregated value for pixels in the first group and a second aggregated value for pixels in the second group.

The method may further comprise:

performing the grouping on a patch of the image to calculate first image features;

dividing the patch into sub-patches; and

calculating further image features on each of the sub-patches.

Detecting corresponding points may comprise computing a binary descriptor representing an image patch surrounding each point and then matching the descriptors of the first and second images by using a Hamming distance.

Computing the binary descriptor may comprise:

performing intensity comparisons between groups of pixels within the patch, wherein a set of 16 patterns are used to define different group of pixels to compare;

dividing the patch into sub-patches; and

performing intensity comparisons likewise on each of the sub-patches.

The method may further comprise determining the patch around an image point that is non-pathologic and remains stationary over time. It is an advantage that the method performs alignment with sub-pixel accuracy.

Obtaining the image objects related to diabetic retinopathy may comprise segmenting microaneurysms by:

normalising a green channel of the image;

applying a threshold on the normalised image to detect candidate features;

removing candidate features based on the size of the candidate features;

applying a rule based method to select candidate features as microaneurysms.

Obtaining image features related to diabetic retinopathy comprises segmenting haemorrhages by:

applying a threshold to obtain a binary mask;

removing blood vessels from the binary mask;

obtaining initial candidate features from the remaining binary mask;

applying a trained machine learning model to classify the candidate features; and

applying a rule based method to remove false positive candidate features.

Obtaining image features related to diabetic retinopathy comprises segmenting exudates by:

applying a threshold to obtain candidate features;

removing false positive candidate features;

applying a trained machine learning model based on pixel-wise features to classify the candidate features;

applying a trained machine learning model based on region-level features to classify the candidate features; and

applying a rule based method to remove false positive candidate features.

The method may further comprise computing a colour difference between images to identify areas on the image that are likely to develop pathology in future. The degree of change may be indicative of the colour difference of the image objects related to diabetic retinopathy present in the first image and the second image.

The method may further comprise normalising colour values between the first image and the second image by reducing colour differences in the colour of the optic disk and vessels between the first image and the second image. Detecting the colour difference may comprise calculating a binary change mask based on a difference in red to green ratio. It is an advantage that the red to green ration is robust against noise.

The method may further comprise classifying image areas within the binary mask based on a change in red or yellow.

The method may further comprise converting the image into an “a” channel and a “b” channel of a CIELAB colour space and the change in red is based on a difference in the “a” channel and a change in yellow is based on a difference in the “b” channel.

It is an advantage that the CIELAB colour channels contain information that allows a more robust colour analysis for red and yellow changes than RGB channels, for example.

Computing the colour difference may comprise a classification of an image area as having a colour difference and the classification is based on neighbouring image areas. The classification may be based on a hidden Markov model random field.

Creating the output representing the calculated numerical pathology score may comprise creating an output image as a predictive map comprising highlighted areas with colour codes of the retina based on the colour difference.

The method may further comprise, before aligning the first image to the second image, correcting illumination of the first image and the second image to enhance the appearances of features.

It is an advantage that illumination correction can reduce the effect of illumination differences between two images that are captured over a relatively long period of time, such as 1-2 years or even longer.

Correcting illumination may comprise:

identifying background areas of the image by identifying areas that are free of any vascular structure, optic disk and objects related to diabetic retinopathy.

identifying and removing both multiplicative and additive shading components of non-uniform illumination from the image.

It is an advantage that performing illumination correction in the proposed fashion does not blur the relevant image objects for diabetic retinopathy which are likely by other illumination correction methods.

A computer system for diagnostic imaging of a retina of a patient with diabetic retinopathy comprises:

an input port to retrieve a first image of the retina captured at a first point in time and to retrieve a second image of the retina captured at a second point in time after the first point in time, the first image being a photographic colour image and the second image being a photographic colour image;

a processor programmed to:
align the first image to the second image to reduce an offset between non-pathologic retina features in the first image and the second image,
obtain image objects related to diabetic retinopathy in the first image and the second image,
calculate a numerical pathology score indicative of a progression of the diabetic retinopathy by calculating a degree of change of the image objects related to diabetic retinopathy between the aligned first and second images, and
create an output representing the calculated numerical pathology score.

### BRIEF DESCRIPTION OF DRAWINGS

A non-limiting example will now be described with reference to the following drawings:

FIG. 1 illustrates a computer system for diagnostic imaging.

FIG. 2 is a schematic illustration of a patient's eye.

FIG. 3 illustrates a method for diagnostic imaging of a retina of a patient with diabetic retinopathy.

FIG. 4 illustrates a system that implements the method of FIG. 3 from a module or object oriented view point.

FIG. 5a shows an example image before correction and FIG. 5b shows the image corrected by the proposed illumination correction technique.

FIG. 6 illustrates a total of 16 different pixel grouping patterns.

FIG. 7a illustrates a patch of 16 pixels and FIGS. 7b and 7c show subsequent divisions of the patch in FIG. 7a.

FIG. 8 illustrates bifurcation points identified in an example image.

FIGS. 9a, 9b and 9c show an exemplary registration by the proposed method where FIG. 9a is the baseline image (“the first image”), FIG. 9b is the follow-up image (“the second image” and FIG. 9c is the mosaic (overlay) image.

FIGS. 10a-c show exemplary pathology segmentation by the proposed method on a single time stamp image. FIG. 10a shows boundaries of the detected microaneurysms, FIG. 10b haemorrhages and FIG. 10c exudates.

FIG. 11 shows an exemplary output report.

FIGS. 12a-d show an exemplary overall change map (colour code image) produced the proposed system where FIG. 12a is the baseline image, FIG. 12b is the follow-up image, FIG. 12c is colour coded overall change image, which is also referred to as change map or predictive map and FIG. 12d is a black and white version of the change map in FIG. 12c.

### DESCRIPTION OF EMBODIMENTS

### Computer System

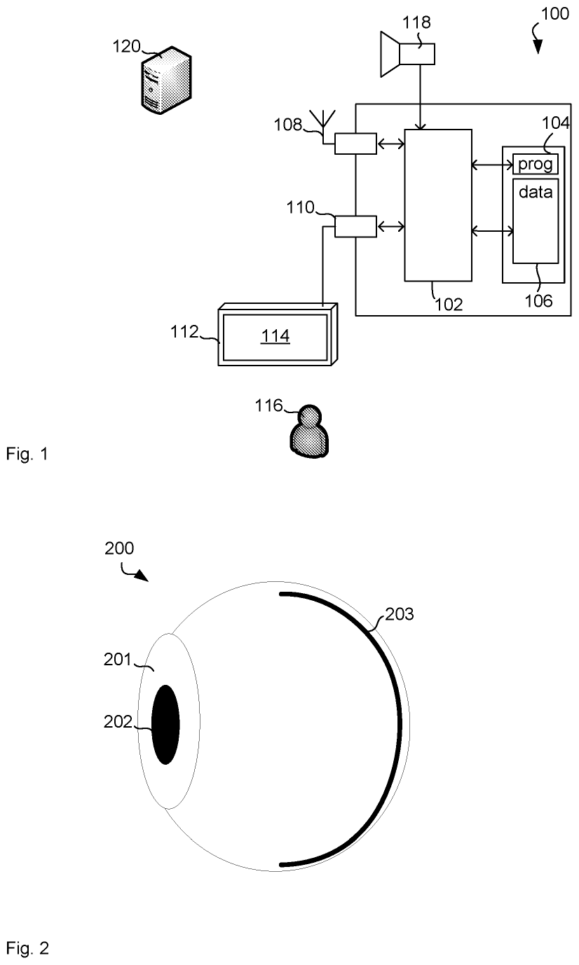

FIG. 1 illustrates a computer system 100 for diagnostic imaging. The computer system 100 comprises a processor 102 connected to program memory 104, data memory 106, a communication port 108 and a user port 110. The user port 110 is connected to a display device 112 that shows data, such as report with a numeric pathology score or a predictive change map 114 to patient 116. The program memory 104 is a non-transitory computer readable medium, such as a hard drive, a solid state disk or CD-ROM. Software, that is, an executable program stored on program memory 104 causes the processor 102 to perform the method in FIG. 2, that is, processor 102 retrieves at least two images captured by a retina camera 118, such as Nidek AFC-210, over time, aligns the images and calculates a pathology score based on pathologic features in the images.

FIG. 2 is a schematic illustration of a patient's 116 eye 200 comprising iris 201, pupil 202 and retina 203. A network of blood vessels (not shown) supply the retina 203 with blood so that the cones and rods can function as light detectors and send a nerve signal representing the detected light to the brain. In the presence of diabetes, the retina 203 typically undergoes pathologic changes which are generally described is diabetic retinopathy (DR). In this disclosure, these changes are detected, quantified and provided to a clinician as a decision support tool or for automatic diagnosis.

Returning back to FIG. 1, the processor 102 may store the images as well as the calculated score on data store 106, such as on RAM or a processor register. Processor 102 may also send the determined score via communication port 108 to a server 120, such as central medical record.

The processor 102 may retrieve data, such as retina images, from camera 118, data memory 106 as well as from the communications port 108 and the user port 110, which is connected to a display 112 that shows a visual representation 114 of the image and/or the pathology score to a user 116. In one example, the processor 102 receives image data from a remote camera via communications port 108, such as by using a Wi-Fi network according to IEEE 802.11. The Wi-Fi network may be a decentralised ad-hoc network, such that no dedicated management infrastructure, such as a router, is required or a centralised network with a router or access point managing the network.

In one example, the processor 102 receives and processes the image data in real time. This means that the processor 102 determines the pathology score every time image data is received from camera 118 and completes this calculation before the camera 118 sends the next image update to create a “live view” of the retina including highlighted areas of pathologic objects.

Although communications port 108 and user port 110 are shown as distinct entities, it is to be understood that any kind of data port may be used to receive data, such as a network connection, a memory interface, a pin of the chip package of processor 102, or logical ports, such as IP sockets or parameters of functions stored on program memory 104 and executed by processor 102. These parameters may be stored on data memory 106 and may be handled by-value or by-reference, that is, as a pointer, in the source code.

The processor 102 may receive data through all these interfaces, which includes memory access of volatile memory, such as cache or RAM, or non-volatile memory, such as an optical disk drive, hard disk drive, storage server or cloud storage. The computer system 100 may further be implemented within a cloud computing environment, such as a managed group of interconnected servers hosting a dynamic number of virtual machines.

It is to be understood that any receiving step may be preceded by the processor 102 determining or computing the data that is later received. For example, the processor 102 processes an image and stores the image in data memory 106, such as RAM or a processor register. The processor 102 then requests the data from the data memory 106, such as by providing a read signal together with a memory address. The data memory 106 provides the data as a voltage signal on a physical bit line and the processor 102 retrieves the image via a memory interface.

It is to be understood that throughout this disclosure unless stated otherwise, nodes, edges, graphs, solutions, variables, images, scores and the like refer to data structures, which are physically stored on data memory 106 or processed by processor 102. Further, for the sake of brevity when reference is made to particular variable names, such as “period of time” or “image objects” this is to be understood to refer to values of variables stored as physical data in computer system 100.

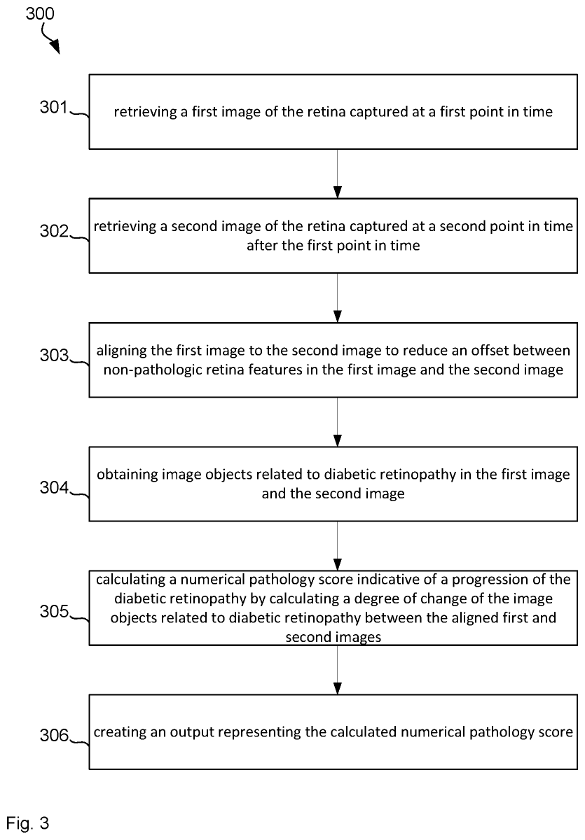

FIG. 3 illustrates a method 300 as performed by processor 102 for diagnostic imaging of a retina of a patient 116 with diabetic retinopathy. FIG. 2 is to be understood as a blueprint for the software program and may be implemented step-by-step, such that each step in FIG. 3 is represented by a function in a programming language, such as C++ or Java. The resulting source code is then compiled and stored as computer executable instructions on non-transitory program memory 104.

It is noted that for most humans performing the method 300 manually, that is, without the help of a computer, would be practically impossible. Therefore, the use of a computer is part of the substance of the invention and allows performing the necessary calculations that would otherwise not be possible due to the large amount of data and the large number of calculations that are involved. More particularly, a human could get a feeling for the progression of the retinopathy by inspecting the images. But it is impossible for most humans to derive a numerical pathology score without the help of computers.

### Method

In one example, patient 116 visits a doctors practice or an eye clinic, looks into a retina camera and the camera captures an image of the retina. The camera then stores the image as an image file or other ways so that it is retrievable by processor 102. The camera 118 may also provide a video stream with a sequence of images. It is noted that throughout this disclosure when reference is made to a first image and a second image, or to a baseline image and a follow-up image, these labels are chosen arbitrarily

After a period of time, such as one year, the patient 116 returns to have another retina image captured and stored. Method 300 then commences by retrieving 301 a first image of the retina captured at the first point in time. Then, processor 102 retrieves 302 the second image of the retina captured at the second point in time after the first point in time. The period of time can be chosen arbitrarily but practically, it should be at a time where changes in the retina could have occurred and where the condition of the patient 116 has not deteriorated too far. For example, one day would likely be too short as the retina would not have changed. On the other hand, 10 years would likely be too long for a patient where retinopathy has been detected in the first image. It is also noted that the second image does not have to be captured by the same camera. In particular, it is possible with the disclosed solution that the image resolution, contrast, sharpness, illumination and other factors vary across subsequent images.

Processor 102 aligns 303 the first image to the second image to reduce an offset between non-pathologic retina features in the first image and the second image. It is noted that this disclosure makes a difference between non-pathologic retina features and image objects related to diabetic retinopathy. In this sense, non-pathologic retina features are those features of the retina that do not typically show a change caused by or associated with retinopathy. These non-pathologic features may be physiologic features and may include the colour or shape of physiologic features. In the examples described herein, the non-pathologic features are blood vessels (i.e. the vascular structure) of the retina. More specifically, non-pathologic features may be the branching points (i.e. bifurcation points) of the blood vessels of the retina. Since the non-pathologic features do not change significantly over time, especially their location within the retina does not change significantly over time, they can serve as robust landmarks when aligning the images by reducing an offset between the non-pathologic features. In other words, processor 102 overlays both images and scales, rotates and shifts the images until the non-pathologic features (e.g., bifurcation points) match. Processor 102 may scale, rotate and shift only the first, the second or both images to perform the alignment. Aligning the images does not necessarily mean storing a new version of the scaled, rotated and shifted image but it may mean storing only the transformation parameters, such as a transformation matrix representing the scaling, rotation and shift. This matrix can then be used whenever processor 102 accesses the modified image. Further detail on the alignment process is provided further below.

Processor 102 then obtains 304 image objects related to diabetic retinopathy in the first image and the second image. It is noted that processor 102 can obtain the image objects in step 304 before or after the alignment in step 303 as these two steps are not dependent on each other. The image objects are objects that may change over time as caused by the diabetic retinopathy. In the examples herein, these objects include microaneurysms, haemorrhages, exudates and intra-retinal micro-vascular abnormalities. Obtaining image objects may comprise applying an object detection algorithm to the image data and determining areas that are covered by the respective image object. This may equally be referred to as “segmenting” the image objects as the areas within the images are segmented for each object. This may equally be described as assigning multiple pixels to an object or determining an association between the multiple pixels to the object. For example, the object may be referenced by an object identifier, such as a sequential integer, and the image coordinates in pixel numbers are stored together with the identifier of the object with which that pixel is associated.

Processor 102 then calculates 305 a numerical pathology score indicative of a progression of the diabetic retinopathy. ‘Numerical’ in this context means that the output is a number value, such as a binary, float or integer. The numerical score can increase or decrease to indicate a respective increase or decrease in the severity of image objects in relation to the retinopathy or vice versa. Processor 102 calculates the score by calculating a degree of change of the image objects related to diabetic retinopathy between the aligned first and second images. The degree of change may be a metric representing a change in colour, shape, size or other physical appearance. The score may be a binary score in the sense that it is Boolean I/O score to indicate change/no-change.

Finally, processor 102 creates 306 an output representing the calculated numerical pathology score. As described further below, the output may be a numerical output of the score, a graphical colour coded map or another output. The output may also be a control output to control another device, such as a notification device or other services that sends a notification via SMS, email or mobile app. The output may be conditional on the score so that the output is only generated when the score meets a pre-defined threshold.

### Modular View

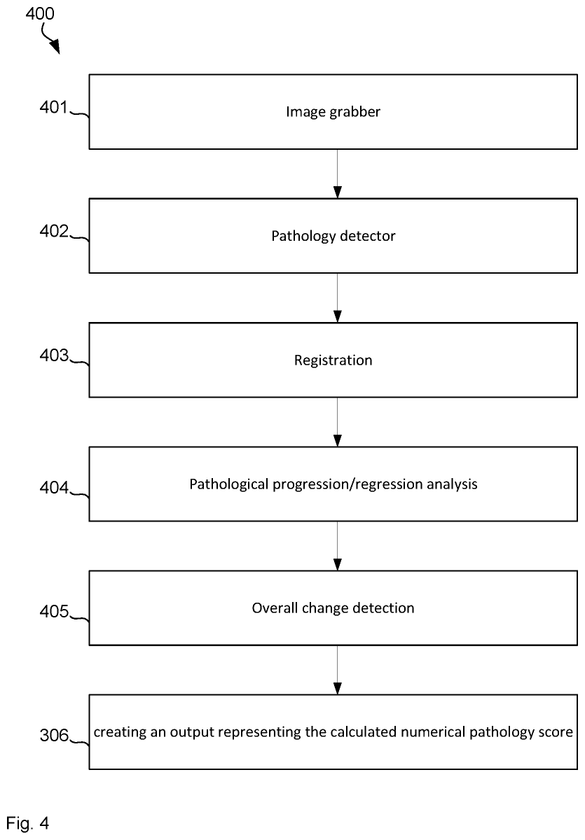

FIG. 4 illustrates a system 400 that implements method 300 from a module or object oriented view point. Each module may be implemented as a class in an object oriented programming language or as separate binaries or computers, virtual machines, cloud instances, compute services (such as Amazon Lambda) or other structures. As such, the modules in FIG. 4 constitute a digital processing pipeline. In particular the proposed system 400 includes an image grabber module 401 (related to steps 301 and 302) that permits importing digital colour fundus images collected at different sites and in different times. In addition with importing images, if available, it also imports several other associated information such as when the image was captured, type of the camera used for the capture, whether the image is of left or right eye, the field and angle of acquisition and whether or not mydriasis was used in the capture.

A pathology detection module 402 (related to step 304) detects and segments the visible pathologies in the image. Prior to detection and segmentation of pathologies, illumination correction of the image is performed. An illumination correction technique is described below and applied to eliminate non-uniform and/or poor illumination without affecting pathology appearance. Machine learning techniques are also described below applied for the segmentation of microaneurysms, haemorrhages and exudates from the image. The pathology detection module 402 also supports human actions to create new outlining, and to change or delete pathology outlining's by the automated method.

A registration module 403 (related to step 303) aligns images and thus establishes pixel-to-pixel correspondence between two or more retinal images from different time, viewpoints and sources. Longitudinal (over time) registration is an important preliminary step to analyse longitudinal changes on the retina including disease progression. For example, microaneurysms may be automatically detected independently in the each of the images collected over time, however, to determine how they are evolving over time or in other words to determine their turn over it is important to map them among images. It is important to note that for longitudinal retinal images potential overlap between images and minimal geometric distortion among them are very common, however, the challenge is the determination of reliable retinal features over time based on which registration can be performed. Relying on the phenomenon that retinal vessels are more reliable over time, a registration method is proposed here. The method aims to accurately match bifurcation and cross-over points between different timestamp images.

A pathological progression/regression analysis module 404 (related to step 305) analyses the changes of the pathologies namely microaneurysms, haemorrhages, and exudates; and provides a change summary. The summary includes number of certain pathology (e.g. microaneurysms, haemorrhages, exudates) that are present in each visit, number of the pathology that are common in both visits, overall change (increase or decrease (in %)) in areas of the pathology that are common in both visits, number of newly formed pathology and number of pathology that are disappeared.

An overall change analysis module 405 (related to step 306) summarises microvascular changes such as changes in artery to vein ratios, changes in central retinal artery equivalent, changes in central retinal vein equivalent, changes in artery and vein tortuosity. The overall change analysis module also detects the increase or decrease of yellowness; and increase or decrease of redness in the follow up image with respect to the base line image. Increase or decrease of yellowness can be associated with formation or disappearance of microaneurysms and/or haemorrhages over time. Likewise increase or decrease of yellowness can be associated with formation or disappearance of exudates. This could easily guide how the patient is responding to treatment. The disclosure below also proposes a method for computing colour difference.

This disclosure provides a system to detect and analyse diabetic related changes in the retina in a more objective and computable manner for quantitative assessment of how the disease is progressing and/or how the patient is responding to treatment over time. The system includes an image grabber module 401, a pathology detection module 402, an image registration module 403, a pathological progression/regression analysis module 404 and an overall change analysis module 405.

Here a detail description of each of the sub-modules used in the system is provided.

### Illumination Correction

Between the image grabber module 401 and the pathology detector module 402 (after steps 301, 302 and before step 303), there may be a further module for non-uniform illumination correction because Fundus images frequently show unwanted variations in brightness due to over-all imperfections in the image acquisition process. Correcting the illumination of the images enhances the appearance of image features, namely the non-pathologic features that are used for image alignment.

Non-uniform/poor illumination across the retina limits the pathological information that can be gained from the image, and thus is corrected prior to pathology detection. Illumination correction is performed in the luminance channel. To compute the luminance or brightness information from the RGB image, HSV colour space transform is used. The image acquisition process of fundus photographs is described by the following model

f=g(f^{o})=S_{M}f^{o}+S_{A}, (1)

where, f is the observed image, g(⋅) represents the acquisition transformation function, f° is the original image, S_{M} and S_{A} are respectively the multiplicative and additive shading component. In this work both multiplicative and additive shading components are estimated, however, one at a time. To estimate S_{A}, it is assumed that the multiplicative shading component is absent and thus equation 1 is simplified to

f=f^{o}+S_{A}

S_{A} is estimated from the observed image based on linear filtering as below

[symbol]

=LPF(f_{background})+C_{A}, (2)

Where LPF denotes a low pass filter, f background is the background image that is free of any vascular structure, optic disk and visible lesions. In this sense, processor 102 identifies background areas of the image by identifying areas that are free of any vascular structure, optic disk and objects related to diabetic retinopathy and identifies and removes both multiplicative and additive shading components of non-uniform illumination from the image.

To compute the background image a preliminary extraction of the pixels belonging to the background set is performed. To compute the following two assumptions about the background pixels are made:
All background pixels have intensity values significantly different than the foreground pixels.
In a neighbourhood of w×w pixels at least 50% of them are background pixels.

An efficient average filter based on integral images is used to realize the low-pass filter in equation (2). While estimating S_{M}, the additive shading component is assumed to zero and thus equation 1 is simplified to f=S_{M}f^{o}·S_{M} is estimated from the observed image based on homomorphic filtering as below

[symbol]

=exp(LPF(log f))·C_{M}, (3)

where, C_{M} represents a multiplicative constant to restore an adequate grey level. Two independent shade free images f′ and f″ are computed relying on equation (2) and (3). The final illumination corrected image is the average of f′ and f″.

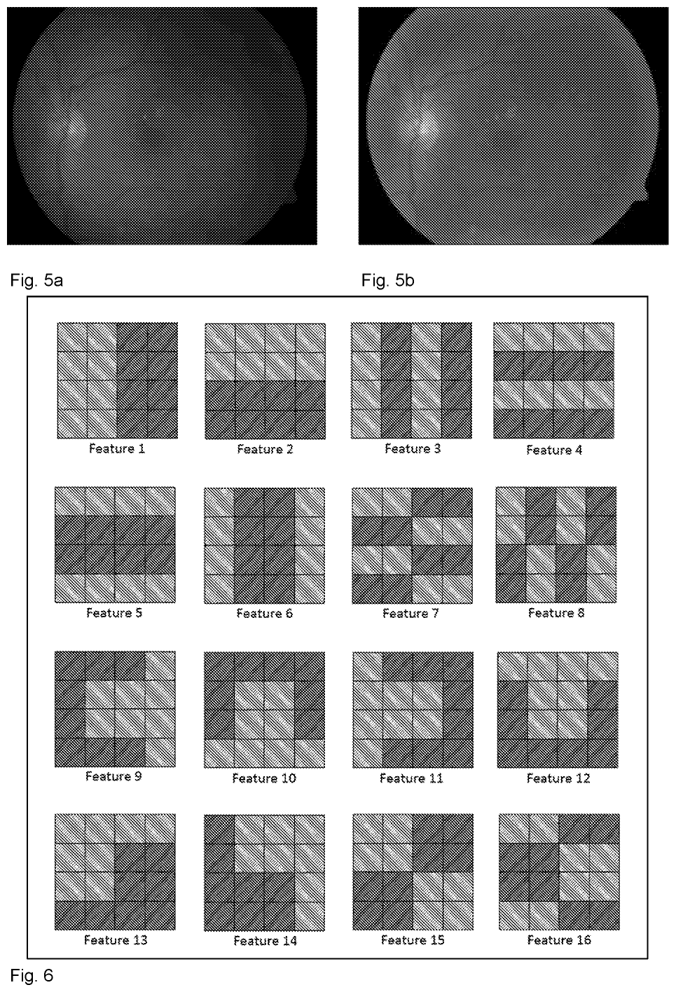

FIG. 5a show an example image before correction and FIG. 5b shows the image corrected by the proposed illumination correction technique.

### Alignment

To compare images that are collected over time, they are registered which is also referred to “alignment” herein as shown in step 303 and module 403. To facilitate registration a descriptor named HARB (HAar features for Retinal Bifurcation points) is used by processor 102 in order to detect corresponding points in the non-pathologic retina features and reducing an offset between the corresponding points.

The registration is performed in two steps. In the first step a preliminary registration is performed relying on Speeded Up Robust Features (SURF) computed on the vasculature and similarity transformation model. SURF is further described in Herbert Bay, Andreas Ess, Tinne Tuytelaars, Luc Van Gool “SURF: Speeded Up Robust Features”, Computer Vision and Image Understanding (CVIU), Vol. 110, No. 3, pp. 346-359, 2008, which is incorporated herein by reference.

In the second step the proposed HARB descriptors are computed on bifurcation points and quadratic transformation model is considered to ensure fine registration relying on accurate matching of bifurcation points. A HARB descriptor relies on a pattern that may comprise two rectangles defining two respective groups of pixels. The pixels from the first rectangle/group are added into a first sum and the pixels from the second rectangle/group are added into a second sum. The descriptor then represents whether the first sum or the second sum is greater. Instead of the sum, other aggregated values can be used, such as average intensity. The proposed HARB descriptor is described in more detail as below.

To describe the bifurcation point a patch P of size 16×16 around the point is considered. That is, the first step preliminary registration determines candidates for non-pathologic features and the second step defines a patch around those candidates and calculates the descriptors for that patch to confirm whether or not it is actually a non-pathologic feature and remains stationary over time and is to be used for alignment. Different groupings of pixels inside the patch is considered and the average intensities of different groups are compared to generate the descriptor. FIG. 6 illustrates a total of 16 different pixel grouping patterns which are reminiscent of Haar features. Each of the 16 features have light grey and dark grey squares. The light grey squares together define the first group of pixels and the dark squares together define the second group of pixels.

More formally, relying of each grouping pattern, 1-bit of the HARB descriptor is compute based on the following test τ:

$$τ ( P ; {p}_{t}^{2} ; {p}_{t}^{2} ) := \{ \begin{matrix}1 & if p ( {p}_{t}^{1} ) < p ( {p}_{t}^{2} ) \\ 0 & else\end{matrix} , \qquad ( 3 )$$

where, p(pₜ¹) and p(pₜ²) are respectively the average intensity of pixels in light and dark grey areas shown in FIG. 6. Thus a 16-bit vector is generated relying on the 16 features.

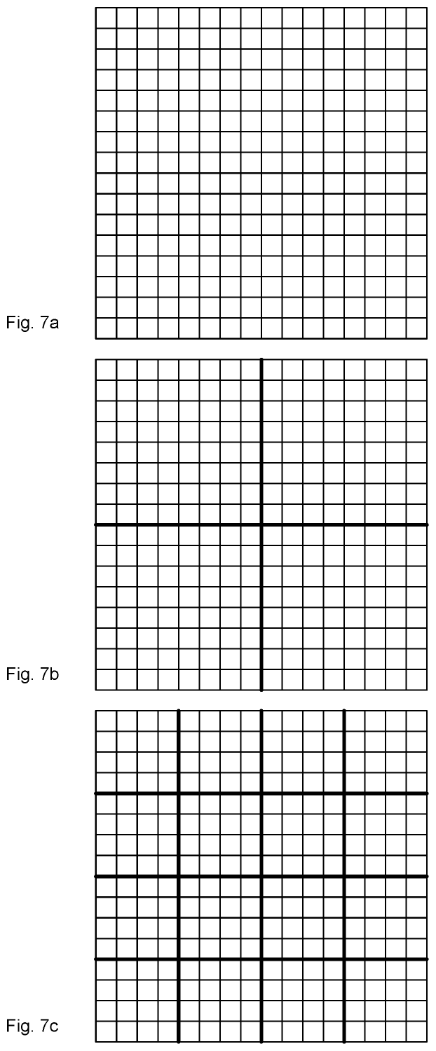

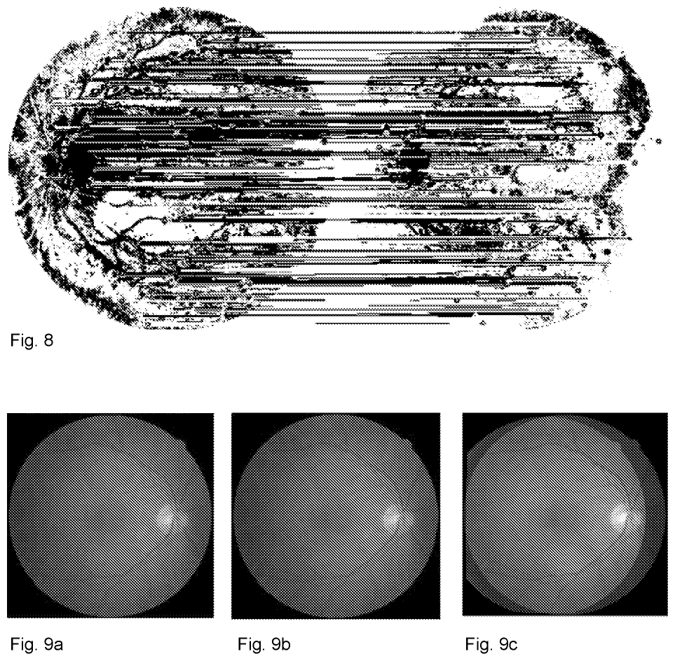

FIG. 7a illustrates a patch of 16 pixels. Then the patch is divided into 4 equal sized sub-patches as shown in FIG. 7b and for each sub-patch such comparisons are performed to generate the bit vector. Instead of using all the 16 features, the first 12 features are used, and thus for each sub-patch in FIG. 7b a 12-bit vector is generated. Each of the sub-patches is further divided into 4 equal sized sub-sub-patches as shown in FIG. 7c and a 12-bit vector is computed for each of them. The total length of HARB descriptor to describe a bifurcation point is 256 bit (=16+4×12+4×4×12). Hamming distance, which counts the number of different bits between two binary strings of same length, is used to measure the similarity between HARB descriptors. The lower the Hamming distance, the more similar they are. FIG. 8 illustrates the identified bifurcation points matching relying on proposed descriptor.

FIGS. 9a, 9b and 9c show an exemplary registration by the proposed method where FIG. 9a is the baseline image (“the first image”), FIG. 9b is the follow-up image (“the second image” and FIG. 9c is the mosaic (overlay) image.

### Object Segmentation

Once baseline and follow-up images are aligned, machine learning methods are applied to segment diabetic retinopathy (DR) pathologies from the images as stated in step 304 and module 402. Specifically microaneurysm, haemorrhages and exudates are segmented. To segment microaneurysms, which typically appears as red dots and are considered as the first DR pathologies, a 4 step method is proposed. The method is summarized as below.
1) The green channel of the image is smoothed and its background image is obtained.
2) The green channel image is normalized based on its background image and further processed by Laplacian of Gaussian filtering.
3) MA candidates are obtained by thresholding operation on the normalized image and removing the candidates with large area such as blood vessels and large haemorrhages.
4) A rule-based method is applied for further examining the properties (compact, contrast and location) of the candidates. The candidates with low compact and contrast values will be removed. The candidates sitting in the optic disc and vessel areas will be removed too. The remaining candidates are considered as microaneurysms.

To segment haemorrhages, which are another sign of diabetic retinopathy and occurs as microaneurysms rupture, a five step method is proposed. The method is summarized as below.
1) A multi-scale Gaussian enhancement process is applied on the green channel to enhance the potential haemorrhage regions. Three recursive Gaussian templates are built and convoluted with the green channels. The minimum value on each pixel location from the three convolved images is chosen as the pixel value for the new generated image, which largely enhances the haemorrhage regions.
2) An adaptive thresholding operation is applied for obtaining HM binary mask (containing HMs and retinal vessels).
3) HM candidates are detected by the operations of removing very large objects (main vessels) and elongated objects (object elongation >threshold, representing vessel fragment) from the above HM and vessel mask.
4). Based on the obtained HM candidates, a random forest (RF) classifier is applied for further true and false HM candidate classification. The RF classifier has been trained on HM labelled images by eye experts by supervised learning. Total 30 parameters are generated from each HM region for the RF classification. The final HMsare identified by the classifier which gives true HM candidate classification.
5) A rule-based processing method is applied to remove false positive HM candidates, such as the candidates in the regions of optic disc and fovea, and then the final HM regions are detected.

To segment exudates (EDs), which are typically formed from leakage of serum proteins, lipids and proteins from retinal blood vessels because of the broken of the vessels, a five step method is proposed.

1) Initial ED candidates are segmented from the illumination corrected green channel by thresholding operation such that pixels above the threshold are kept as ED candidates.
2) In this step, the ED candidates, located at the optic disc region and high reflection regions at the rim of the retinal field which are detected in advance, are considered as false positive EDs and removed.
3) A Random Forest classifier has been built on 50 trees and trained on 23 features from RGB and HSL channels, based on the ED labelled images from experts, for ED pixel-level classification. The Random Forest model is then applied for identifying the ED candidates from above step by pixel-wise. The step will remove a large amount of false positive lesion pixels. Tiny candidates (<4 pixels) and elongated ones along the main vessels are classified as reflections and removed too.
4) A Random Forest classifier with 500 trees has been built, based on 57 features from each region from the images labelled by experts, for ED region-based classification. The pre-trained Random Forest model is applied for identifying true ED regions on the candidates obtained from the above step.
5) A rule-based method is applied for removing the small size and independent bright regions along the vessels and in the nerve fibre regions which are prone to be false positive EDs; and the final ED regions are obtained.

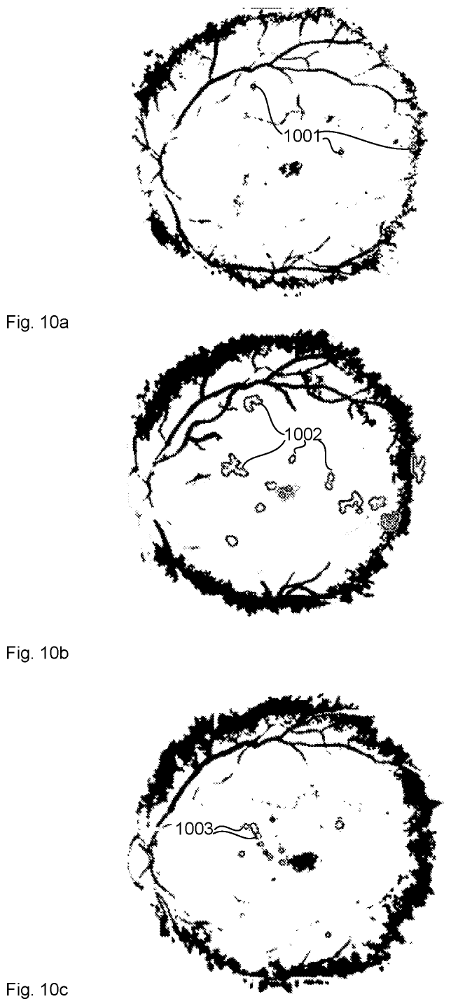

FIGS. 10a-c show exemplary pathology segmentation by the proposed method on a single time stamp image. FIG. 10a shows boundaries of the detected microaneurysms 1001, FIG. 10b haemorrhages 1002 and FIG. 10c exudates 1003 (not all regions are connected with the corresponding reference numeral).

### Change Report

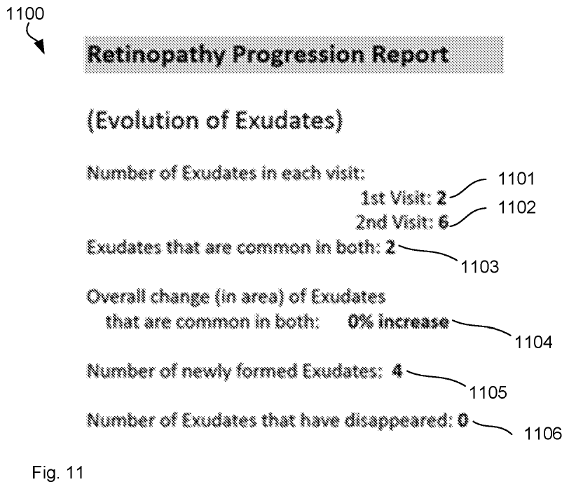

Once pathologies are segmented in baseline and follow-up images they are compared and a detail change analysis report is produced. For each of the pathology types the system outputs the number of that pathology in each visit, number of the common pathology, overall changes in area of the pathology, number of newly formed pathology and finally the number of the pathology that disappeared over time. FIG. 11 shows an exemplary output 1100 created by processor 102 performing method 300. The report in FIG. 11 represents the calculated numerical pathology score, which may include one or more of the following: Number of exudates (i.e. pathologic objects) obtained in the first image 1101 and in the second image 1102, exudates that are common in both images 1103, overall change in area of exudates that are common in both 1104, number of newly formed exudates 1105 and the number of exudates that have disappeared 1106. Other numerical scores may equally be presented.

### Change Map

As an alternative or in addition to the output shown in FIG. 11, processor 102 can also produce an output representing the pathological score graphically, such as to highlight in the retina image where the changes occurred that are reported in FIG. 11. Such a ‘map’ representation is also said to be an output representing the calculated numerical pathology score. Even further, the calculated numerical pathology score may be more than the change in area or number of objects as shown in FIG. 11 but may also extend to change in colour of the image objects, which indicates areas on the image that are likely to develop pathology in future.

Following pathological change analysis between baseline and follow-up images, an overall change analysis between images is performed. Prior to computing the overall changes between images a colour normalization is performed. The colour normalization minimizes the intra subject colour variability between fundus images that are captured at different times. In particular, processor 102 reduces the colour differences in the colour of the non-pathologic features, such as the optic disk and vessels, between the images. In one example, colour correction is performed on the follow-up images but may be performed on the baseline image instead. In essence, if there are more than two images, any image can be used as the baseline image and the other images are colour corrected. It is not essential that the colour in the images is the true colour as perceived by a human observer. Instead, it is sufficient that colour changes are identified accurately, which means the relative colour change should be accurate. For an extreme example, it is possible that the baseline image has a green tinge and in that case, all follow-up images are adjusted to also have green tinge because this preserves the colour difference between the image. In other words, processor 102 performs a relative colour correction that is, in a sense, calibrated by the baseline image.

To perform the correction, mean RGB values of the optic disk and blood vessels of the baseline and follow-up images are computed. Let (m_{OD}^{R}, m_{OD}^{G}, m_{OD}^{B}) and (m_{V}^{R}, m_{V}^{G}, m_{V}^{B}) are the mean RGB values of the optic disk and blood vessels respectively. Then the RGB values of each pixel i of the follow-up image F, corrected using the following formula.

$${R}_{c} = \frac{1}{2} ( \frac{{m}_{{OD}_{I}}^{R}}{{m}_{{OD}_{F}}^{R}} + \frac{{m}_{{V}_{I}}^{R}}{{m}_{{V}_{F}}^{R}} ) \times R , \\ {G}_{c} = \frac{1}{2} ( \frac{{m}_{{OD}_{I}}^{G}}{{m}_{{OD}_{F}}^{G}} + \frac{{m}_{{V}_{I}}^{G}}{{m}_{{V}_{F}}^{G}} ) \times G , \\ {B}_{c} = \frac{1}{2} ( \frac{{m}_{{OD}_{I}}^{B}}{{m}_{{OD}_{F}}^{B}} + \frac{{m}_{{V}_{I}}^{B}}{{m}_{{V}_{F}}^{B}} ) \times B . \qquad ( 5 )$$

Here (m_{OD}^{R}_{I}, m_{OD}^{G}_{I}, m_{OD}^{B}_{I}) and (m_{OD}^{R}_{F}, m_{OD}^{G}_{F}, m_{OD}^{B}_{F}) are the mean RGB values of the optic disk of the baseline and follow-up images respectively. Likewise, (m_{V}^{R}_{I}, m_{V}^{G}_{I}, m_{V}^{B}_{I}) and (m_{V}^{R}_{F}, m_{V}^{G}_{F}, m_{V}^{B}_{F}) are the mean RGB values of the blood vessels of the baseline and follow-up images respectively.

To segment the optic disk first Canny mask is applied for the detection of edges and finally Hough transform is applied for the detection of circle that define optic disk.

To segment the blood vessels at each pixel position of the image, a window of size W×W pixels is considered and the average grey level I_{avg}^{w} is computed. Twelve lines of length L pixels oriented at 12 different directions and passing through the centred pixels are considered and the average of grey levels of pixels along each line is computed. The line with the maximum value Iₘₐₓ^{L} is determined and is called ‘winning line’. Iₘₐₓ^{L} is computed for different values of L, 1≤L≤W; and then compute the generalized line detector defined below.

I_{W}^{L}=Iₘₐₓ^{L}−I_{avg}^{w}. (6)

The responses computed at different scales are finally combined as below.

$${I}_{combined} = \frac{1}{{n}_{L} + 1} ( {\sum }_{L} {I}_{W}^{L} + {I}_{igc} ) . \qquad ( 7 )$$

Here n_{L} is the number of scales used, and I_{igc} is the value of the inverted green channel at the corresponding pixel. Prior to computing I_{combined} standardization of the values of the raw response image is performed to enhance contrast of the image. Once the colour is normalized, the ratio images are computed for each the images. The green and red channel of the image is used to compute the ratio image which is defined mathematically as below.

$${I}_{ratio} = \frac{{I}_{Red}}{{I}_{Green}} . \qquad ( 8 )$$

The ratio image gives additional robustness to noise and illumination artefacts. The ratio image is computed for the images collected at different times, t₁ and t₂, and the difference of the ratio images are calculated.

ΔIᵣₐₜᵢₒ=I_{ratio_t2}−I_{ratio_t1}. (9)

The significant changes are then detected based on the difference image. Assuming a Gaussian distribution for the difference values, a binary change mask, B is computed by comparing the normalized sum of square of the differences within a neighbourhood w, as described by Aach et al.

$${Ω}_{i , j} = \frac{1}{{σ}_{n}^{2}} {\sum }_{( i , j ) ∈ w} Δ {{I}_{ratio} ( i , j )}^{2} , \qquad ( 10 )$$

where σₙ is the noise standard deviation of the difference in the no-change region.

$${B}_{i , j} := \{ \begin{matrix}1 & {Ω}_{i , j} > Γ \\ 0 & otherwise\end{matrix} . \qquad ( 11 )$$

The threshold Γ is derived from the fact that Ω_{i,j} follows a χ² distribution with d=w×w degrees of freedom.

The change mask obtained in the previous step is classified into multiple categories to reflect pigmentation changes relevant to diabetic retinopathy. Table 1 below lists the five classes of interest along with their significance. Each pixel of the change mask is classified into one of these five classes, {Cₗ}_{l=1,2,3,4,5}.

TABLE 1
Pigmentation changes and their significance to diabetic retinopathy.

| Type of colour change | Significance |
|---|---|
| Increase in redness | Appearance of bleeding/ |
| | microaneurysms/haemorrhages or likely |
| | to develop these pathologies in due |
| | course |
| Decrease in redness | Disappearance of bleeding/ |
| | microaneurysms/haemorrhages |
| Increase in yellowness | Appearance of exudates or likely to |
| | develop the pathology in due course |
| Decrease in yellowness | Disappearance of exudates |
| No change | No change |

First the colour normalised images I_{norm_t1}, I_{norm_t2} are transformed into CIELAB colour space. The chroma channels (i.e. a and b) are used to compute the colour difference between images. Let aₜ₁, bₜ₁ are the chroma channels of I_{norm_t1}; and aₜ₂, bₜ₂ are the chroma channels of I_{norm_t2}. Increase, decrease or no change in redness is determined based on the following criteria.

Increase in redness:=aₜ₂(i,j)−aₜ₁(i,j)>T₁

Decrease in redness:=aₜ₁(i,j)−aₜ₂(i,j)>T₁ (12)
No change:=otherwise.
   Here, T₁ is a predefined threshold (e.g. 5).

These pixel level classifications are performed only for the pixel locations that are identified as significant in the binary change mask (i.e. B_{i,j}==1).

Similarly increase, decrease or no change in yellowness is determined based on the following condition.

Increase in yellowness:=bₜ₂(i,j)−bₜ₁(i,j)>T₂

Decrease in yellowness:=bₜ₁(i,j)−bₜ₂(i,j)>T₂ (13)
No change:=otherwise,
   where, T₂ is a predefined threshold (e.g. 7).

When a single pixel satisfies multiple conditions (e.g. increase/decrease in redness as well as increase/decrease in yellowness) one single class is assigned to it based on the priority criterion defined below.

Increase in yellowness>increase in redness>decrease in yellowness>decrease in redness>no change.

The pixel level classification obtained in the previous step can be noisy. It may be likely that a pixel belonging to a particular class C₁ is likely to be surrounded by pixels belonging to the same class. To embed this contextual information into the classification process hidden Markov model random field (HMRF) approach has been used. Given a chromaticity image X, where X_{i,j}=(a_{i,j}, b_{i,j})^{T} is the chromaticity value of pixel (i,j), processor 102 infers a configuration of labels C where C_{i,j}∈{C₁, C₂, C₃, C₄, C₅} denote the class level for X_{i,j}. The conditional distribution of the class level C_{i,j} is modelled as below using Markov assumption

$$P ( {C}_{i , j} \vert \{ {C}_{u , v} , ( u , v ) ∈ N \} ) = \frac{1}{z} exp [ - {H}_{i , j} ] , \qquad ( 8 )$$

where N denotes a small neighbourhood around the pixel, Z is a normalizing factor and H_{i,j} is the Gibbs' energy function defined as below.

H_{i,j}=Σ_{(u,v)∈N}αδₗ(C_{i,j},C_{u,v}). (9)

Here, the parameter α controls the effect of spatial-contextual and is empirically set to

$$\frac{1.5}{{d}^{2}} ,$$

where d is the Euclidean distance from the pixel of interest to its neighbours. The term δₗ is set to −1 when C_{i,j}=C_{u,v}, or 0 otherwise.

According to the maximum-a-posteriori (MAP) criterion, we seek the labelling C* which satisfies

$${C}^{*} = arg {max}_{C} \{ {U}_{data} ( X \vert C , Θ ) + {U}_{context} ( C ) \} . \qquad ( 12 )$$

Here, P(C) is the prior probability defined in eq. (8) and P(X|C, Θ) is the joint likelihood probability defined as below.

P(X|C,Θ)=ΠᵢΠⱼP(X_{i,j}|C,Θ)

=ΠᵢΠⱼP(X_{i,j}|C_{i,j},θ_{i,j}), (11)

where P(X_{i,j}|C_{i,j}, θ_{i,j}) is a Gaussian distribution with parameters θ_{i,j}=(μ_{i,j}, σ_{i,j}). We employed expectation maximization (EM) to estimate the parameter set Θ={θₗ|l∈L}.

In the EM algorithm processor 102 solves C* that minimizes the total posterior energy

$${C}^{*} = arg {max}_{C} \{ P ( X \vert C , Θ ) P ( C ) \} . \qquad ( 10 )$$

Here, U_{context} is a measure of inter-pixel dependency and U_{data} represents the likelihood of a pixel belonging to particular class. U_{context} is derived based on eq. (8) as U_{context}=−H, and U_{data} in the Gaussian case is defined as

U_{data}(X|C,Θ)=ΣᵢΣⱼ[½(X_{i,j}−μ_{i,j})^{T}Σ_{C}_{_{i,j}}⁻¹(X_{i,j}−μ_{i,j})+½ ln|Σ_{C}_{_{i,j}}|],

where Σ_{C}_{_{i,j}} is the co-variance matrix.

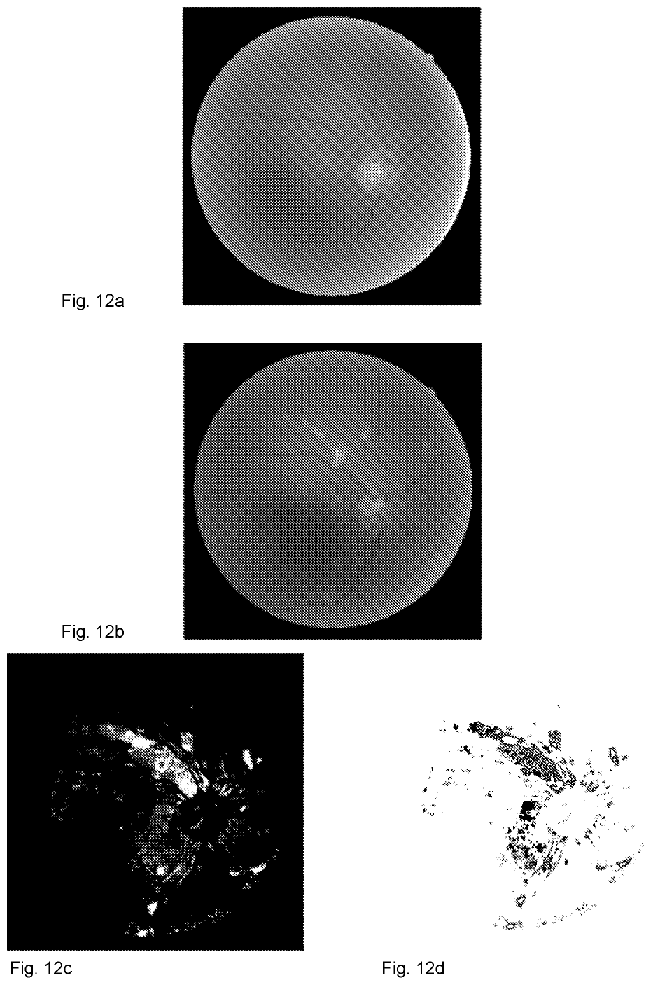

Five different colour codes are used to represent classified pixels. Table 2 details the colour codes and their associated levels. FIGS. 12a-d show an exemplary overall change map (colour code image) produced the proposed system where FIG. 12a is the baseline image, FIG. 12b is the follow-up image, FIG. 12c is colour coded overall change image, which is also referred to as change map or predictive map and FIG. 12d is a black and white version of the change map in FIG. 12c.

TABLE 2
Pigmentation changes and their display colour code.

| Type of colour change | Display colour code |
|---|---|
| Increase in redness | Red |
| Decrease in redness | Blue |
| Increase in yellowness | Yellow |
| Decrease in yellowness | Green |
| No change | Black |

It will be appreciated by persons skilled in the art that numerous variations and/or modifications may be made to the specific embodiments without departing from the scope as defined in the claims.

It should be understood that the techniques of the present disclosure might be implemented using a variety of technologies. For example, the methods described herein may be implemented by a series of computer executable instructions residing on a suitable computer readable medium. Suitable computer readable media may include volatile (e.g. RAM) and/or non-volatile (e.g. ROM, disk) memory, carrier waves and transmission media. Exemplary carrier waves may take the form of electrical, electromagnetic or optical signals conveying digital data steams along a local network or a publically accessible network such as the internet.

It should also be understood that, unless specifically stated otherwise as apparent from the following discussion, it is appreciated that throughout the description, discussions utilizing terms such as “estimating” or “processing” or “computing” or “calculating”, “optimizing” or “determining” or “displaying” or “maximising” or the like, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

The present embodiments are, therefore, to be considered in all respects as illustrative and not restrictive.

1. A method for diagnostic imaging of a retina of a patient with diabetic retinopathy, the method comprising:
retrieving a first image of the retina captured at a first point in time, the first image being a photographic colour image;
retrieving a second image of the retina captured at a second point in time after the first point in time, the second image being a photographic colour image;
aligning the first image to the second image to reduce an offset between non-pathologic retina features in the first image and the second image;
obtaining image objects related to diabetic retinopathy in the first image and the second image;
calculating a numerical pathology score indicative of a progression of the diabetic retinopathy by calculating a degree of change of the image objects related to diabetic retinopathy between the aligned first and second images; and
creating an output representing the calculated numerical pathology score.

2. The method of claim 1, wherein the degree of change is indicative of a change in the number of image objects related to diabetic retinopathy.

3. The method of claim 1, wherein the degree of change is indicative of a change in the area covered by image objects related to diabetic retinopathy present in the first image and the second image.

4. The method of claim 3, further comprising identifying areas on the image that are likely to develop pathology in future by determining a colour difference between the first image and the second image.

5. The method of claim 3, wherein the output comprises a predictive map with colour codes representing areas on the second image that showed changes in colour in comparison to the first image, the colour map being indicative of which areas are likely to develop pathology in future.

6. The method of claim 1, wherein aligning the first image to the second image comprises detecting corresponding points in the non-pathologic retina features and reducing an offset between the corresponding points.

7. The method of claim 6, wherein detecting corresponding points comprises calculating image features for the first and second images by grouping, for each image feature, pixels into a first group and a second group and calculating the image features based on a first aggregated value for pixels in the first group and a second aggregated value for pixels in the second group.

8. The method of claim 7, wherein the method further comprises:
performing the grouping on a patch of the image to calculate first image features;
dividing the patch into sub-patches; and
calculating further image features on each of the sub-patches.

9. The method of claim 7, wherein detecting corresponding points comprises computing a binary descriptor representing an image patch surrounding each point and then matching the descriptors of the first and second images by using a Hamming distance.

10. The method of claim 9, computing the binary descriptor comprises:
performing intensity comparisons between groups of pixels within the patch, wherein a set of 16 patterns are used to define different group of pixels to compare;
dividing the patch into sub-patches; and
performing intensity comparisons likewise on each of the sub-patches.

11. The method of claim 8, wherein the method further comprises determining the patch around an image point that is non-pathologic and remains stationary over time.

12. The method of claim 1, wherein obtaining the image objects related to diabetic retinopathy comprises performing one or more of the following (a) or (b) or (c):
(a) segmenting microaneurysms by:
normalising a green channel of the image;
applying a threshold on the normalised image to detect candidate features;
removing candidate features based on the size of the candidate features;
applying a rule based method to select candidate features as microaneurysms; or
(b) segmenting haemorrhages by:
applying a threshold to obtain a binary mask;
removing blood vessels from the binary mask;
obtaining initial candidate features from the remaining binary mask;
applying a trained machine learning model to classify the candidate features; and
applying a rule based method to remove false positive candidate features; or
(c) segmenting exudates by:
applying a threshold to obtain candidate features;
removing false positive candidate features;
applying a trained machine learning model based on pixel-wise features to classify the candidate features;
applying a trained machine learning model based on region-level features to classify the candidate features; and
applying a rule based method to remove false positive candidate features.

13-14. (canceled)

15. The method of claim 1, wherein
the method further comprises computing a colour difference between images to identify areas on the image that are likely to develop pathology in future and
the degree of change is indicative of the colour difference of the image objects related to diabetic retinopathy present in the first image and the second image.

16. (canceled)

17. The method of claim 16, wherein the method further comprises:
normalising colour values between the first image and the second image by reducing colour differences in the colour of the optic disk and vessels between the first image and the second image.

18. The method of claim 17, wherein
computing the colour difference comprises calculating a binary change mask based on a difference in red to green ratio, and
the method further comprises:
classifying image areas within the binary mask based on a change in red or yellow; and
converting the image into an “a” channel and a “b” channel of a CIELAB colour space, the change in red being based on a difference in the “a” channel and a change in yellow being based on a difference in the “b” channel.

19-20. (canceled)

21. The method of claim 15, wherein
computing the colour difference comprises a classification of an image area as having a colour difference and the classification is based on neighbouring image areas and
the classification is based on a hidden Markov model random field.

22. (canceled)

23. The method of claim 15, wherein creating the output representing the calculated numerical pathology score comprises creating an output image as a predictive map comprising highlighted areas with colour codes of the retina based on the colour difference.

24. The method of claim 1,
wherein the method further comprises, before aligning the first image to the second image, correcting illumination of the first image and the second image to enhance the appearances of features, and
wherein correcting illumination comprises:
identifying background areas of the image by identifying areas that are free of any vascular structure, optic disk and objects related to diabetic retinopathy; and
identifying and removing both multiplicative and additive shading components of non-uniform illumination from the image.

25. (canceled)

26. A non-transitory computer readable medium with software code stored thereon that, when executed by a computer, causes the computer to perform the method of claim 1.

27. A computer system for diagnostic imaging of a retina of a patient with diabetic retinopathy, the computer system comprising:
an input port to retrieve a first image of the retina captured at a first point in time and to retrieve a second image of the retina captured at a second point in time after the first point in time, the first image being a photographic colour image and the second image being a photographic colour image;
a processor programmed to:
align the first image to the second image to reduce an offset between non-pathologic retina features in the first image and the second image,
obtain image objects related to diabetic retinopathy in the first image and the second image,
calculate a numerical pathology score indicative of a progression of the diabetic retinopathy by calculating a degree of change of the image objects related to diabetic retinopathy between the aligned first and second images, and
create an output representing the calculated numerical pathology score.
